(19) United States
(12) Patent Application Publication
(10) Pub. No.: US 2025/0154236 A1
(43) Pub. Date: May 15, 2025

(54) PREVENTION AND TREATMENT OF HEADACHES

(71) Applicants: GlaxoSmithKline Intellectual Property (No. 3) Limited, Stevenage (GB); 23andMe, Inc., South San Francisco, CA (US)

(72) Inventors: Gareth Trevor YOUNG, Phoenixville, PA (US); Caixia JIA, Devon, PA (US); Wenjie REN, Berwyn, PA (US); Dominic Samuel BERNS, Pacifica, CA (US); Adam Mohamed NAGUIB, Redwood City, CA (US)

(21) Appl. No.: 18/832,579

(22) PCT Filed: Jan. 30, 2023
(86) PCT No.: PCT/IB2023/050788

§ 371 Date: Jul. 24, 2024

Related U.S. Application Data
(60) Provisional application No. 63/304,958, filed on Jan. 31, 2022.

(51) Int. Cl. C07K 16/22; A61K 39/00; A61P 25/06

(52) U.S. Cl. CPC C07K 16/22; A61P 25/06; A61K 2039/505; C07K 2317/76; C07K 2317/92

(57) ABSTRACT

The present disclosure relates to use of artemin inhibitors in prevention or treatment of headaches such as migraine.

### CROSS-REFERENCE TO RELATED APPLICATIONS

This application claims the benefit of U.S. provisional application No. 63/304,958 filed on Jan. 31, 2022, which is incorporated herein by reference in its entirety.

### SEQUENCE LISTING

This application contains a Sequence Listing, which has been submitted electronically in computer readable form in XML file format which is hereby incorporated herein by reference in its entirety. Said XML file, created on Jan. 20, 2023, is named “70131WO01_SL” and is 4,517 bytes in size.

### FIELD OF THE INVENTION

The present disclosure relates to a use of, or a method of administering, an inhibitor of human artemin for preventing and/or treating a headache such as migraine or trigeminal neuralgia. In some cases, the use/method disclosed herein prevents and/or treats migraine. The use/method herein can aim at least one new target, artemin (ARTN), which is a member of GDNF ligand family and involved in nervous system development and postnatal neural plasticity, and effectively reduces migraine frequency in migraine patients (e.g., patients who do not respond to CGRP inhibitors) by preventing the impact of artemin on trigeminal ganglion neuron sensitization. In some instances, the use/method herein reduces release of migraine-associated neurotransmitters such as CGRP (a peptide released by peripheral neurons including somatosensory neurons of the dorsal root, vagal and trigeminal ganglia), and other neuropeptides associated with migraine for example Substance P (SP) which can induce dural mast cell degranulation and neurogenic inflammation that potentially contributes to migraine, and impact migraine-associated trigeminal ganglia (TG) neuron physiology.

### BRIEF SUMMARY

In one aspect, provided herein is an inhibitor of human artemin for use in the prevention or treatment of migraine, or a method of preventing or treating migraine in a subject in need thereof, comprising administering an inhibitor of human artemin to the subject.

### BRIEF DESCRIPTION OF THE DRAWINGS

FIG. 1 is a LocusZoom plot for the association between migraine defined using the ‘migraine_diagnosis’ classification and genetic variants at the ARTN locus, after conditioning on the primary signal in the region.

FIG. 2 is a diagram showing ARTN (transcript ENST00000372354.3) in its predicted cDNA (top line, SEQ ID NO:3) sequence and resulting amino acid protein sequence (bottom line, SEQ ID NO: 2).

FIGS. 3A to 3D are a set of line charts showing that artemin potentiated capsaicin (CAP) and allyl isothiocyanate (AITC)-stimulated CGRP release from adult rat trigeminal ganglia neurons.

FIGS. 4A to 4D are a set of graphic charts demonstrating that an anti-artemin antibody and a RET (Receptor tyrosine kinase, a co-receptor of artemin) inhibitor blocked artemin's effect on potentiated CGRP release from adult rat trigeminal ganglia (TG) neurons.

FIG. 5A and FIG. 5B demonstrate that artemin dose-dependently increased capsaicin (CAP) and allyl isothiocyanate (AITC)-stimulated Substance P (SP) release from rat TG neurons.

FIG. 6A and FIG. 6B demonstrate that a mouse anti-artemin antibody (30 nM) and a RET inhibitor (100 nM) completely blocked artemin-potentiated Substance P (SP) release from both capsaicin (CAP) and allyl isothiocyanate (AITC)-stimulated TG neurons.

FIG. 7A and FIG. 7B demonstrate that artemin potentiated capsaicin (CAP) and allyl isothiocyanate (AITC)-stimulated Pituitary adenylate-cyclase-activating polypeptide (PACAP) release from rat TG neurons.

FIG. 8 demonstrates that local dural infusion of recombinant rat artemin in rat significantly increased periorbital sensitivity to mechanical stimulation, a representative migraine phenotype in a preclinical migraine model.

### DETAILED DESCRIPTION

The present disclosure provides a use of, or a method of administering, an artemin inhibitor (e.g., anti-artemin antibody) to prevent and/or treat headache disorders such as migraine. In some instances, the term migraine refers to a primary headache disorder that satisfies the diagnostic criteria according to the International Classification of Headache Disorders (ICHD). In some instances, the migraine is a migraine without aura, migraine with aura, hemiplegic migraine, retinal migraine, chronic migraine, menstrual migraine, vestibular migraine, status migrainosus, probable migraine, or any combination of the foregoing. In some cases, a use/method herein prevents and/or reduces a symptom of migraine, including head pain, visual disturbances, photophobia, phonophobia, nausea, or vomiting.

In some cases, a use or method disclosed herein can prevent and/or treat trigeminal neuralgia. In some instances, trigeminal neuralgia involves trigeminal ganglion (TG) neuron sensitization and elevates calcitonin gene-related peptide (CGRP) and other neuropeptides level in blood, cerebrospinal fluid (CSF), and plasma of trigeminal neuralgia (TN) patients, and a use/method herein with an artemin inhibitor can treat and/or prevent the trigeminal neuralgia, given the potential role of artemin in TG neuron sensitization and CGRP release.

In some cases, a use or method disclosed herein can prevent and/or treat cluster headache. In some instances, cluster headache is induced by elevated CGRP in blood, or CGRP levels are elevated in cluster headache, and a use/method herein with an artemin inhibitor can treat and/or prevent the cluster headache, given the potential role of artemin in TG neuron sensitization and CGRP release.

In some cases, a use or method disclosed herein can prevent and/or treat post-traumatic headache. In some instances, post-traumatic headache is induced or exacerbated by an increase of CGRP levels in blood, or CGRP levels are elevated in post-traumatic headache, and a use/method herein with an artemin inhibitor can treat and/or prevent the post-traumatic headache, given the potential role of artemin in TG neuron sensitization and CGRP release.

In some cases, a use or method disclosed herein can prevent and/or treat temporomandibular disorder (TMD). TMD can be a complex set of head and facial conditions affecting the muscles of mastication and/or the temporomandibular joint (TMJ) which may present with pain of myofacial or arthrogenic origin. In some instances, CGRP is released in response to TMJ pain or orofacial pain, and a use/method herein with an artemin inhibitor can treat and/or prevent the cluster headache, given the role of CGRP release in pain associated TMD.

In some cases, a use or method disclosed herein can prevent and/or treat chronic pain disorders such as fibromyalgia. In some instances, a subject having fibromyalgia may have increased CGRP release and suffer moderate to severe headaches, and a use/method herein with an artemin inhibitor can treat and/or prevent the fibromyalgia.

In some instances, a method of preventing or treating migraine in a subject (e.g., a human patient) in need thereof comprises administering an effective amount of an artemin inhibitor to the subject, whereby the migraine is prevented or treated, optionally where the method further comprises identifying a subject prone to or suffering migraine before the administration.

In some instances, the present disclosure provides a method of reducing CGRP level in a subject (e.g., a human patient) in need thereof comprising administering an effective amount of an artemin inhibitor to the subject, whereby the CGRP level is reduced.

### Inhibitor of Human Artemin

In some cases, an inhibitor disclosed herein is a small molecule (molecular weight <1000 Daltons), nucleic acid molecule (e.g., oligonucleotides, nucleic acid aptamer, RNA, DNA, siRNA), a peptide, a peptide aptamer, a polypeptide, a protein, or an antibody (e.g., human or humanized monoclonal antibody antagonizing artemin), or an alternative antibody format thereof, or a fragment thereof (e.g., comprising an antigen binding domain). In some instances, the inhibitor is a monoclonal antibody, e.g., a humanized monoclonal antibody. In some instances, the inhibitor is a bispecific antibody targeting (e.g., antagonizing) CGRP and human artemin. In some instances, the inhibitor is a bispecific antibody targeting (e.g., antagonizing) GFRα3 (glial cell-line derived neurotrophic factor receptor alpha-3) and human artemin. In some instances, the inhibitor is a bispecific antibody targeting (e.g., antagonizing) Substance P and human artemin. In some instances, the inhibitor is present in an aqueous solution, optionally for subcutaneous administration.

In some cases, a human artemin inhibited or blocked by an inhibitor disclosed herein comprises an amino acid sequence set out in SEQ ID NO:1 or SEQ ID NO:2, or at least: 80%, 85%, 90%, 95%, 96%, 97%, 98%, or 99% identical to SEQ ID NO:1 or SEQ ID NO:2. In some cases, a use/method herein is for a patient such as a human subject. In some instances, the human subject is detected to having an artemin comprising the sequence set out in SEQ ID NO:1.

The term “antibody” is used herein in the broadest sense to refer to molecules with an immunoglobulin-like domain (for example IgG, IgM, IgA, IgD or IgE) and includes monoclonal antibodies, recombinant antibodies, synthetic antibodies, polyclonal antibodies, chimeric antibodies, human antibodies, humanised antibodies, multispecific antibodies (e.g., bispecific antibodies), and heteroconjugate antibodies; a single variable domain, antigen binding antibody fragments (e.g., Fab, F(ab′)₂, Fv, disulphide linked Fv, single chain Fv, disulphide-linked scFv, diabodies, TANDAB™, etc.) and modified versions of any of the foregoing.

The term “domain” refers to a folded protein structure which retains its tertiary structure independent of the rest of the protein. Generally, domains are responsible for discrete functional properties of proteins and in many cases may be added, removed or transferred to other proteins without loss of function of the remainder of the protein and/or of the domain. The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain that is capable of binding an antigen or epitope independently of a different variable region or domain may be referred to as a “domain antibody” or “dAb™”. A single variable domain may be a human single variable domain, but also includes single variable domains from other species such as rodent, nurse shark and Camelid VHH dAbs™. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised, and such domains may be “single variable domains”. As used herein VH includes camelid VHH domains. Alternative antibody formats are those where the CDRs are arranged onto a suitable non-immunoglobulin protein scaffold or skeleton. The non-immunoglobulin scaffold may be a derived from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; LDL receptor class A domains; EGF domains; scorpion toxin kunitz type domains of human protease inhibitors; and fibronectin/adnectin.

In some cases, an inhibitor of human artemin reduces CGRP levels in serum or plasma (e.g., measured from cranial blood or blood from an antecubital or cubital vein), for example by at least 10% (e.g., at least 15%, at least 20%, at least 25%, at least 30%, at least 35%, at least 40%, at least 45%, at least 50%, at least 60%, at least 70%, at least 80% or at least 90%) than a corresponding blocking concentration of a CGRP inhibitor, e.g., erenumab, fremanezumab, galcanezumab, eptinezumab, ubrogepant, rimpegepant, or atogepant, or a GFRα3 antagonist, e.g., REGN5069. In some instances, the CGRP levels are measured from cubital blood.

### Prophylactic or Therapeutic Use/Method

In some aspects, the present disclosure provides a use of, or a method of administering, an inhibitor of human artemin in the prevention and/or treatment of migraine.

In some cases, the use/method is for the prevention of migraine, e.g., chronic migraine. In some instances, prevention of migraine can refer to the situation where the reduction in mean migraine duration or occurrences is greater for a population of patients administered with the inhibitor of artemin compared to a placebo or a therapeutic agent directly targeting CGRP (e.g., a CGRP inhibitor such as erenumab, fremanezumab, galcanezumab, eptinezumab, ubrogepant, rimpegepant, or atogepant) or a therapeutic agent directly targeting GFRα3 such as GFRα3 antagonist, e.g., REGN5069. In some instances, the reduction in mean migraine-occurrence days weekly or monthly in patients administered with the inhibitor of artemin is at least 1 day, at least 2 days, at least 3 days, at least 4 days, at least 5 days, or at least 6 days. In some instances, the reduction in mean migraine-occurrence weeks over one month in patients administered with the inhibitor of artemin is at least 1 week, at least 2 weeks, or at least 3 weeks. In some instances, the reduction in mean migraine-occurrence months over one year in patients administered with the inhibitor of artemin is at least 1, at least 2, at least 3, at least 4, at least 5, at least 6, at least 7, at least 8, at least 9, at least 10, or at least 11 months.

In some instances, prevention of migraine refers to the situation where the 50% responder rate is higher (e.g., at least 5% higher, at least 10% higher, at least 15% higher, at least 20% higher, at least 25% higher, at least 30% higher, at least 35% higher, at least 40% higher, at least 45% higher, or at least 50% higher) for a population of patients administered with the inhibitor of artemin compared to a placebo or a therapeutic agent directly targeting CGRP (e.g., a CGRP inhibitor such as erenumab, fremanezumab, galcanezumab, eptinezumab, ubrogepant, rimpegepant, or atogepant) or a therapeutic agent directly targeting GFRα3 such as GFRα3 antagonist, e.g., REGN5069. In some instances, the inhibitor of human artemin is administered in combination with at least one other (i.e., at least a second) therapeutic agent selected from: botulinum toxin A, a CGRP inhibitor, an anticonvulsant, an antihypertensive, a β-blocker, an antidepressant and a non-steroidal anti-inflammatory drug. In some instances, the inhibitor of human artemin is administered in combination with at least one other (i.e., at least a second) therapeutic agent selected from: valproate, divalproex sodium, amitriptyline, topiramate, venlafaxine, metoprolol, propranolol and timolol.

In some cases, the use/method is for the treatment of migraine. In some instances, treatment of migraine refers to the symptomatic treatment of acute migraine. Migraines may be present with or without aura or visual disturbances. In some instances, the use/method herein treats migraine with aura or visual disturbances. In some instances, the use/method herein treats migraine without aura or visual disturbances.

In some instances, treatment of migraine refers to the situation where a percentage of patients that are pain free 2 hours after administration of the inhibitor of human artemin is higher for a population of patients administered with the inhibitor of human artemin compared to a population of patients administered with a placebo or a therapeutic agent directly targeting CGRP or GFRα3. Being “pain free” can be a patient reported measure. In some instances, the percentage of patients that are pain free 2 hours after administration of the inhibitor of human artemin is higher (e.g., at least 5% higher, at least 10% higher, at least 15% higher, at least 20% higher, at least 25% higher, at least 30% higher, at least 35% higher, at least 40% higher, at least 45% higher, or at least 50% higher) compared to a placebo or a therapeutic agent directly targeting CGRP, e.g., a CGRP inhibitor such as erenumab, fremanezumab, galcanezumab, eptinezumab, ubrogepant, rimpegepant, or atogepant, or a therapeutic agent directly targeting GFRα3 such as GFRα3 antagonist, e.g., REGN5069.

In some instances, treatment of migraine refers to the situation where a percentage of patients that have no headache pain 2 hours after administration of the inhibitor of human artemin and have no relapse of headache pain within 24 hours after administration of the inhibitor of human artemin is higher compared to a population of patients administered with a placebo or a therapeutic agent directly targeting CGRP or GFRα3. Headache pain can be a patient reported measure. In some instances, the percentage of patients that have no headache pain 2 hours after administration of the inhibitor of human artemin and have no relapse of headache pain within 24 hours after administration of the inhibitor of human artemin is higher (e.g., at least 5% higher, at least 10% higher, at least 15% higher, at least 20% higher, at least 25% higher, at least 30% higher, at least 35% higher, at least 40% higher, at least 45% higher, or at least 50% higher) compared to a placebo or a therapeutic agent directly targeting CGRP, e.g., a CGRP inhibitor such as erenumab, fremanezumab, galcanezumab, eptinezumab, ubrogepant, rimpegepant, or atogepant, or a therapeutic agent directly targeting GFRα3 such as GFRα3 antagonist, e.g., REGN5069.

In some instances, treatment of migraine refers to the situation where a percentage of patients that have no headache pain 2 hours after administration of the inhibitor of human artemin and have no relapse of headache pain within 48 hours after administration of the inhibitor of human artemin is higher compared to a population of patients administered with a placebo or a therapeutic agent directly targeting CGRP or GFRα3. Headache pain can be a patient reported measure. In some instances, the percentage of patients that have no headache pain 2 hours after administration of the inhibitor of human artemin and have no relapse of headache pain within 48 hours after administration of the inhibitor of human artemin is higher (e.g., at least 5% higher, at least 10% higher, at least 15% higher, at least 20% higher, at least 25% higher, at least 30% higher, at least 35% higher, at least 40% higher, at least 45% higher, or at least 50% higher) compared to a placebo or a therapeutic agent directly targeting CGRP, e.g., a CGRP inhibitor such as erenumab, fremanezumab, galcanezumab, eptinezumab, ubrogepant, rimpegepant, or atogepant, or a therapeutic agent directly targeting GFRα3 such as GFRα3 antagonist, e.g., REGN5069.

In some cases, the inhibitor of human artemin is administered in combination with at least one other (i.e., at least a second) therapeutic agent that is: acetaminophen (or an acetaminophen containing composition), a triptan, an ergot alkaloid (e.g., dihydroergotamine, ergotamine), an opioid receptor antagonist (e.g., naltrexone, naloxone, nalmefene, samidorphan), a non-steroidal anti-inflammatory drug, a butalbital containing product, an anti-emetic, caffeine, dexamethasone, ubrogepant, lasmiditan, any pharmaceutically acceptable salt thereof, or any combination thereof. In some instances, the inhibitor of human artemin is administered in combination with acetaminophen. In some instances, the inhibitor of human artemin is administered in combination with an ergot alkaloid, for example dihydroergotamine. In some instances, the inhibitor of human artemin is administered in combination with an opioid receptor antagonist, for example naltrexone. In some instances, the inhibitor of human artemin is administered in combination with a triptan, for example selected from the group consisting of: almotriptan, eletriptan, frovatriptan, naratriptan, rizatriptan, sumatriptan, zolmitriptan, any pharmaceutically acceptable salt thereof, and any combination thereof. In some instances, the inhibitor of human artemin is administered in combination with sumatriptan or any pharmaceutically acceptable salt thereof (e.g., sumatriptan succinate). In some instances, the inhibitor of human artemin is administered in combination with oral sumatriptan at a maximum dose of 200 mg/day. In some instances, the inhibitor of human artemin is administered in combination with a non-steroidal anti-inflammatory drug, for example acetylated salicylates (aspirin), non-acetylated salicylates (diflunisal, salsalate), propionic acids (naproxen, ibuprofen), acetic acids (diclofenac, indomethacin), enolic acids (meloxicam, piroxicam), anthranilic acids (meclofenamate, mefenamic acid), naphthylalanine (nabumetone), selective COX-2 inhibitors (celecoxib, etoricoxib), etodolac, fenoprofen, flurbiprofen, ketorolac (e.g., tromethamine salt), ketoprofen, oxaprozin, sulindac, tolmetin, any pharmaceutically acceptable salt thereof, or any combination thereof. In some instances, the inhibitor of human artemin is administered in combination with a non-steroidal anti-inflammatory drug selected from the group consisting of diclofenac, ibuprofen, naproxen, ketolorac, any pharmaceutically acceptable salt thereof, and any combination thereof. In some instances, the inhibitor of human artemin is administered in combination with an anti-emetic selected from the group consisting of: promethazine, prochlorperazine, metoclopramide, trimethobenzamide, ondansetron, any pharmaceutically acceptable salt thereof, and any combination thereof. In some instances, exemplary pharmaceutically acceptable salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate. In some instances, exemplary pharmaceutically acceptable salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In some instances, the inhibitor of human artemin and the other therapeutic agent(s) may be administered together or separately and, when administered separately, administration may occur simultaneously or sequentially, in any order. Simultaneous administration may be achieved by administration of (1) a unitary pharmaceutical composition including the therapeutic agents; or (2) simultaneous administration of separate pharmaceutical compositions each including one of the therapeutic agents. Alternatively, the combination may be administered separately in a sequential manner wherein one treatment agent is administered first and the other second or vice versa. Such sequential administration may be close in time or remote in time.

In some instances, the present disclosure provides a method for decreasing the level of calcitonin gene related peptide (CGRP) in cubital blood in a subject comprising administering a therapeutically effective amount of an inhibitor of human artemin as defined herein to the subject; whereby the CGRP level in cubital blood of the subject is decreased. In some instances, the present disclosure provides a method for decreasing the level of calcitonin gene related peptide (CGRP) in cranial blood in a subject comprising administering a therapeutically effective amount of an inhibitor of human artemin as defined herein to the subject; whereby the CGRP level in cranial blood of the subject is decreased. In some instances, the present disclosure provides a method for decreasing calcitonin gene related peptide (CGRP) level in the systemic circulation of a subject comprising administering a therapeutically effective amount of an inhibitor of human artemin as defined herein to the subject; whereby the CGRP level in the systemic circulation of the subject is decreased.

### Pharmaceutical Compositions/Routes of Administration/Dosages

An inhibitor of human artemin may be administered by an effective route. In some instances, the inhibitor of human artemin may be administered by an injection for example subcutaneously or intravenously.

According to one aspect, provided herein is a pharmaceutical composition comprising an inhibitor of human artemin and a pharmaceutically acceptable excipient. According to another aspect, the present disclosure provides a process for the preparation of a pharmaceutical composition comprising admixing an inhibitor of human artemin with a pharmaceutically acceptable excipient.

Pharmaceutical formulations may be presented as units such as solutions or suspensions in aqueous or non-aqueous liquids; edible foams or whips; or oil-in-water liquid emulsions or water-in-oil liquid emulsions. In some instances, the inhibitor is present in an aqueous solution.

Pharmaceutical formulations adapted for injection include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The present disclosure also provides unitary pharmaceutical compositions in which the inhibitor of human artemin and one or more other therapeutic agent(s) disclosed herein may be administered in combination, concurrently or sequentially.

### EXAMPLES

### Example 1: Genome Wide Association Study in Individuals with Migraine Shows Artemin (and its Variants) Contribute to Migraine Pathophysiology in Humans

Genome-wide association study (GWAS) of individuals diagnosed with migraine identified a genetic region in the ARTN locus reaching the accepted genome-wide statistical threshold of p<5×10⁻⁸ on human chromosome 1.

### GWAS Analysis

Association test results were computed for the genotyped and the imputed SNPs. Association testing was performed using logistic regression, assuming additive allelic effects. For tests using imputed data, the imputed dosages were used rather than best-guess genotypes. Covariates were included for age, sex, and the top five principal components to account for residual population structure. The association test P value was computed using a likelihood ratio test, which in our experience is better behaved than a Wald test on the regression coefficient. In order to avoid issues relating to machine precision, association P values were truncated to a minimum value of 2.2×10-308, prior to genomic inflation correction. All individuals included in the analyses provided informed consent and answered surveys online according to our human subject protocol, which was reviewed and approved by Ethical & Independent Review Services, a private institutional review board (www.eandireview.com).

### Genotyping and SNP Imputation

DNA extraction and genotyping were performed on saliva samples by CLIA-certified and CAP-accredited clinical laboratories of Laboratory Corporation of America. Samples were genotyped on one of five genotyping platforms. The V1 and V2 platforms were variants of the Illumina HumanHap550+BeadChip and contained a total of about 560,000 SNPs, including about 25,000 custom SNPs. The V3 platform was based on the Illumina OmniExpress+BeadChip and contained a total of about 950,000 SNPs and custom content to improve the overlap with our V2 array. The V4 platform is a fully custom array and includes a lower redundancy subset of V2 and V3 SNPs with additional coverage of lower-frequency coding variation, and about 570,000 SNPs. The V5 platform, in current use, is an Illumina Infinium Global Screening Array of about 640,000 SNPs supplemented with about 50,000 SNPs of custom content. Samples that failed to reach 98.5% call rate were re-analyzed. Individuals whose analyses failed repeatedly were re-contacted to provide additional samples. A total of 1,522,458 variants have been genotyped across the five genotyping platforms.

The imputation panel combines two independent reference panels: the Human Reference Consortium (HRC) panel and a smaller reference panel. The HRC data were from the European Genome-Phenome Archive at the European Bioinformatics Institute (accession EGAD00001002729). The HRC data includes 27,165 samples. Variants were liftovered to hg38 and excluded if their new positions were on a different chromosome. Variants were then re-phased using SHAPEIT4 (odelaneau.github.io/shapeit4/). Finally, singletons were excluded. The final HRC reference panel included 27,165 samples and 39,057,040 SNPs (no insertions or deletions (indels)). For the smaller reference panel, 12,217 samples were selected from multiple internal and external whole genome sequencing datasets. The cohort composition is:

| Cohort name | Origin | Samples | Approximate ancestry |
|---|---|---|---|
| AFAM | Internal | 2,389 | African American |
| AJ | Internal | 553 | Ashkenazi Jewish |
| Parkinson's cohort | Internal | 2,721 | European |
| Latino | Internal | 1,961 | Latino |
| LRRK2 carriers | Internal | 303 | European |
| 1000G¹ | External | 2,493 | Diverse |
| CHM syndip² | External | 1 | European |
| GTEx v8³ | External | 838 | Diverse (USA) |
| HGDP⁴ | External | 770 | Diverse |
| SGDP⁵ | External | 188 | Diverse |
| Total | | 12,217 | |

¹The 1000 Genomes Project Consortium.
²A synthetic-diploid benchmark for accurate variant-calling evaluation.
³The GTEx Consortium atlas of genetic regulatory effects across human tissues.
⁴Insights into human genetic variation and population history from 929 diverse genomes.
⁵The Simons Genome Diversity Project: 300 genomes from 142 diverse populations.

The criteria used for including samples in the panel were the following:
consented for research as of 2020 Jan. 18;
depth>median (depth)−3*MAD (median absolute deviation depth); (this criteria was applied within each cohort);
contamination <0.05; (estimated by verifybamid);
<0.05 chimeric reads;
median insert size >=250 bp;
r² with genotyping array >=0.8;
>=3 million SNPs called;
All samples passed these criteria with the exception of GTEx v8 samples which were sometimes slightly outside these bounds. Their inclusion was forced due to their value in eQTL mapping.

All samples were aligned and duplicate marked using one of two very similar pipelines. Reads were aligned to GRCh38_full_analysis_set_plus_decoy_hla.fa (ftp.1000genomes.ebi.ac.uk/voll/ftp/technical/reference/GRCh38_reference_genome/GRCh38_f ull_analysis_set_plus_decoy_hla.fa). For recent sequencing datasets (after 2019 Jan. 1), it combines bwa mem 0.7.15-r1140 alignment, Picard MarkDuplicates 2.15.0, and BQSR with GATK 4.beta.5 (github.com/CCDG/Pipeline-Standardization/blob/master/PipelineStandard.md). For older datasets (before 2019 Jan. 1), the data were re-aligned using an in-house pipeline which consisted of bwa mem 0.7.15-r1140 alignment, duplicate marking with samblaster v0.1.24, and no BQSR.

Variants were called in each individual sample using DeepVariant-0.8.0 (github.com/google/deepvarian) to produce GVCFs. The GVCFs were then joint-called using GLnexus-1.2.3 (github.com/dnanexus-rnd/Glnexus). The following quality controls were applied to variants:
singletons were removed,
genotypes with GQ<20 were set to missing,
variants with >20% missingness (after the GQ filter) were removed,
variants with >30% excess heterozygosity were removed.

Finally, variants were phased using SHAPEIT4. It is worth noting that SHAPEIT4 imputed missing genotypes and produced a final panel without missingness. The smaller reference panel included 12,217 samples and 82,078,539 variants (73,852,355 SNPs+8,226,184 indels).

The HRC panel and the smaller panel were merged into an imputation panel that contained 85,099,656 unique variants, as shown in the table below:

| | | The smaller | | |
|---|---|---|---|---|
| Allele count | HRC only | panel only | Intersect | Total |
| 2 | 1,637 | 361,322 | 65 | 363,024 |
| 3 | 169,613 | 12,332,326 | 16,801 | 12,518,740 |
| 4 | 252,754 | 6,870,553 | 61,391 | 7,184,698 |
| 5 | 639,821 | 4,356,185 | 173,225 | 5,169,231 |
| >5 | 1,957,292 | 22,122,230 | 35,784,441 | 59,863,963 |
| all | 3,021,117 | 46,042,616 | 36,035,923 | 85,099,656 |

More than 36 million variants were found in both the HRC panel and the smaller panel, 46 million in the smaller panel only, and 3 million in HRC panel only. It is worth noting that the imputation panel does not include singletons and only doubletons with functional importance were retained. Using Beagle 5, variant imputation was performed separately for the three sets of variants (HRC only, smaller panel only, and intersect), using three distinct sets of reference samples. For each participant, 85 million variants were imputed after determining the participant ancestry, and selecting and phasing high-quality genotyped variants within each ancestry. Because participants were genotyped on one of the five genotyping platforms (v1 to v5), the imputation is performed independently for each genotyped platform.

### Conditional Analysis

Conditional analysis was used as a tool to identify secondary association signals at a GWAS locus, involving association analysis conditioning on the primary associated SNP at the locus to test whether there are any other SNPs significantly associated. The approach starts with the top associated SNP (index SNP), followed by a stepwise procedure of selecting additional SNPs, one by one, according to their conditional P values. The stepwise procedure stops when the conditional P>10⁻⁵. The conditional analysis only uses imputed data.

### Results

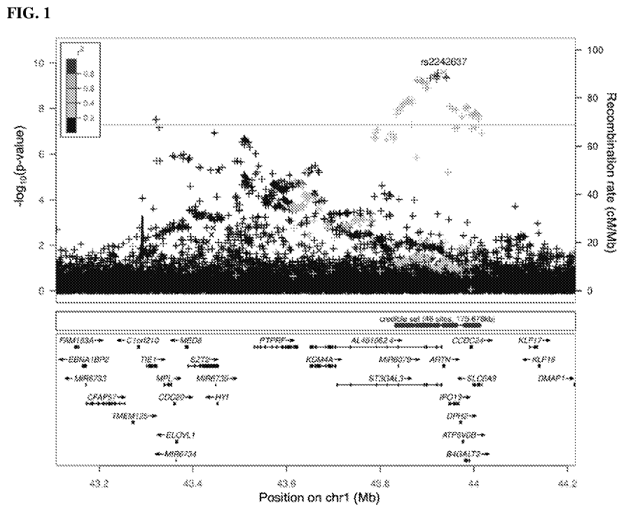

An independent association was identified between migraine diagnosis and several SNPs near the ARTN gene. The sentinel SNP from the second step of conditional analysis for migraine associations in this region is the missense SNP rs2242637 (P=2.4×10⁻¹⁰, OR=1.031). FIG. 1 is a regional association plot for the secondary conditional signal for migraine diagnosis on chromosome 1 where the x-axis shows physical positions on human genome build GRCh38/hg38 and the left hand y-axis shows the −log 10 of the p-value for association with migraine diagnosis. Each point in the depicted plots represents a genetic variant tested for association in the region. The grey horizontal line represents the genome-wide significance threshold of 5×10⁻⁸. Human genes in the region are depicted on the lower panel. These GWAS data indicate that the locus (genetic region) shown is implicated in susceptibility to migraine diagnosis in humans.

In FIG. 1, the trait analyzed was “migraine diagnosis” where “the cases” are individuals with a migraine diagnosis and “the controls” are individuals who did not have a migraine diagnosis. The x-axis displays position on chromosome 1 using GRCh38/hg38 as the human reference genome build. The left hand y-axis is the −log₁₀ (p-value) from a logistic regression testing for an association between migraine case-control status and genotype. Plus symbols (+) denote variants for which genotype was imputed; circle symbols (o) denote variants for which genotype calls were used; x symbols (x) denote imputed coding variants; diamond symbols (⋄) denote genotyped coding variants. Color reflects the linkage disequilibrium (calculated as r2) between the lead SNP (shown in grey) and other nearby SNPs. The grey horizontal line represents the genome-wide significance threshold of 5×10⁻⁸. The credible set track displays the number and location of variants in the 99% credible set, which is likely to contain the causal variant. The gene track displays genes in the locus with thick bars representing exons and thin lines representing introns.

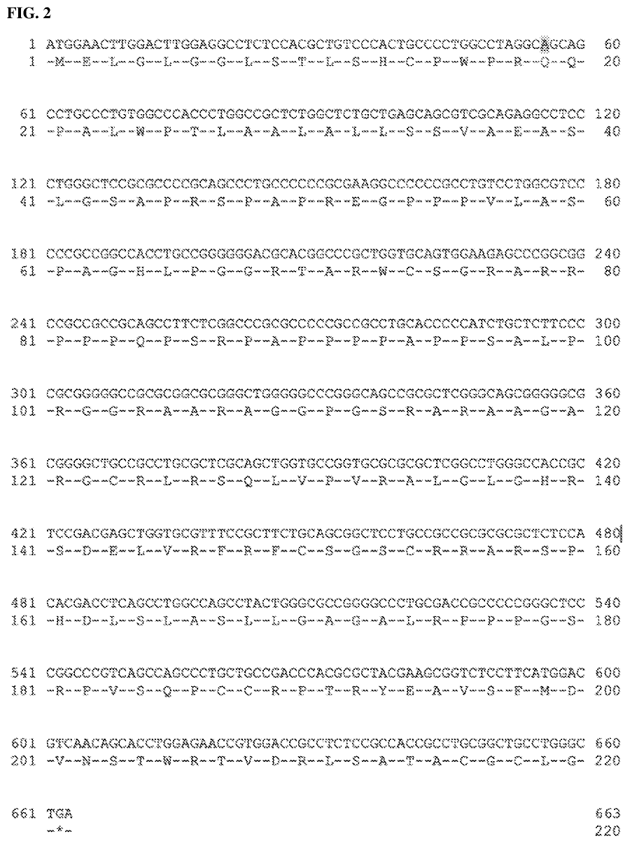

A common genetic variant changing the amino acid sequence of the ARTN protein was found to be the sentinel SNP. This variant rs2242637 (dbSNP build 153 identifier) is located on chromosome 1 at position 43935712 (of the human genome build GRCh38/hg38) and the observed frequency of the migraine diagnosis risk allele “G” is 0.93 in the research participant population with predominantly European ancestry (the protective allele “A” has a frequency of 0.07). This variant can also be described at the amino acid level in ARTN, for example: Gln19Arg, ENST00000372354.3 (FIG. 2, coding sequence SEQ ID NO:3 and corresponding amino acid sequence SEQ ID NO:2 for the 19Q isoform of ARTN). In FIG. 2, nucleotide/amino acid numbering is displayed at the beginning/end of each row. Rs2242637 (highlighted), the SNP associated with migraine diagnosis frequency in the GWAS, encodes amino acid 19 in this transcript and can result in a Gln (A nucleotide-underlined in FIG. 2) or Arg (G nucleotide) amino acid incorporation into the ARTN protein.

The GWAS analysis identified the rs2242637 missense SNP in the ARTN coding region as the SNP in this locus with the lowest P value. Consequently, these data imply that ARTN has the highest probability of being the gene that is functionally responsible for the association between this locus and migraine diagnosis. Therefore, these new GWAS data demonstrated that the ARTN protein and its functions contribute to migraine pathophysiology in humans. These genetic data also indicated that genotyping of individuals at rs2242637 may identify individuals with an improved probability of responding to modulation of ARTN function as a treatment for migraine.

### Example 2. Anti-Artemin Antibodies Ability to Block Artemin-Potentiated Migraine-Associated Neuropeptides Release

The present disclosure demonstrates artemin's impact on migraine-associated neuropeptides release including CGRP (Calcitonin Gene-Related Peptide), PACAP (Pituitary Adenylate Cyclase-Activating Polypeptide) and SP (Substance P) from TG neurons stimulated by different triggers including CAP and AITC. In some cases, artemin can potentiate neuropeptide release from stimulated TG neurons, anti-artemin antibody and RET inhibitor block this potentiation and bring the neuropeptides level back to control level (stimulation alone group), supporting that neutralizing artemin decreases multiple neuropeptides level and would have therapeutic benefit for migraine and trigeminal neuralgia and other indications disclosed herein.

### Rat Trigeminal Ganglia (TG) Neuron Culture and Neuropeptides (CGRP, Substance P and PACAP) ELISA Assays:

TG Cultures were prepared from 8-10-week Sprague-Dawley rats. Following cervical dislocation and cardiac cessation, trigeminal ganglia (TG) were dissociated and collected in ice cold HBSS buffer. TGs were digested in 20 U/ml papain for 20 min followed by another 20-min enzymatic digestion in 2% Collagenase Type 4 and Dispase II in 37° C. water bath. Digested TG ganglia were suspended in Leibovitz's L15 medium containing 10% Fetal Bovine Serum, 1% penicillin/streptomycin, 18.5 mM NaHCO₃, 38 mM glucose. Cell suspensions were filtered in a 70 μm then a 40 μm cell strainer and were layered onto Leibovitz's L15 medium containing 4% BSA cushion followed by slow speed centrifuge at 100 g, 10 min. Cell pellets were suspended with DMEM medium containing 10% Fetal Bovine Serum and 1% penicillin/streptomycin. Cells were plated onto 96-well plates and cultured for 24 hours at 37° C., 5% CO₂. Recombinant rat artemin protein and vehicle control buffer were added into appropriate wells for 24-hour pre-incubation as needed. For experiments testing the effect of an anti-artemin antibody or RET inhibitor, these agents were added to the TG culture simultaneously with artemin for 24 h for CGRP and SP assay or 48 h for PACAP assay. After 24 h or 48 h incubation, cell culture medium was removed and assay buffer containing TRPV1 agonist capsaicin (CAP) or TRPA1 agonist allyl isothiocyanate (AITC) were added into wells and incubated at 37° C., 10 min to trigger neuropeptide release. At the end of stimulation, the supernatant was collected and CGRP and SP content were measured using ELISA assay kits (Bertin Bioreagent). For PACAP assay, cells were cultured and pre-incubated with artemin for 48 hours prior to agonist stimulation to trigger PACAP release. PACAP content in supernatant was measured using an in-house developed ELISA assay.

### Artemin Dural Infusion Study in Rat:

Briefly, three different doses of rat artemin protein, inflammatory soup cocktail and vehicle buffer were administered daily via a stainless-steel cannula (22GA, Plastics One) embedded onto dura for 5 consecutive days to produce persistent facial hypersensitivity. Sensory testing occurred during the day using von Frey filaments with reproducible calibrated buckling forces varying from 0.4-10 g utilizing the simplified Chaplan up-and-down method. Allodynia was tested by perpendicularly touching the periorbital region causing slight buckling of the filament for approximately 5 seconds. The periorbital withdrawal threshold (g) was calculated based on the response pattern and the force of the final filament. The periorbital threshold data was recorded on the side of artemin, Inflammatory Soup Cocktail, or vehicle infusion only.

### Results:

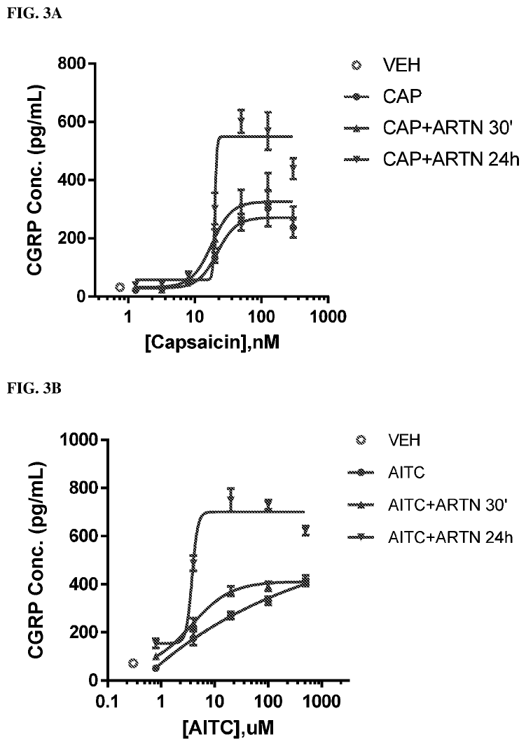

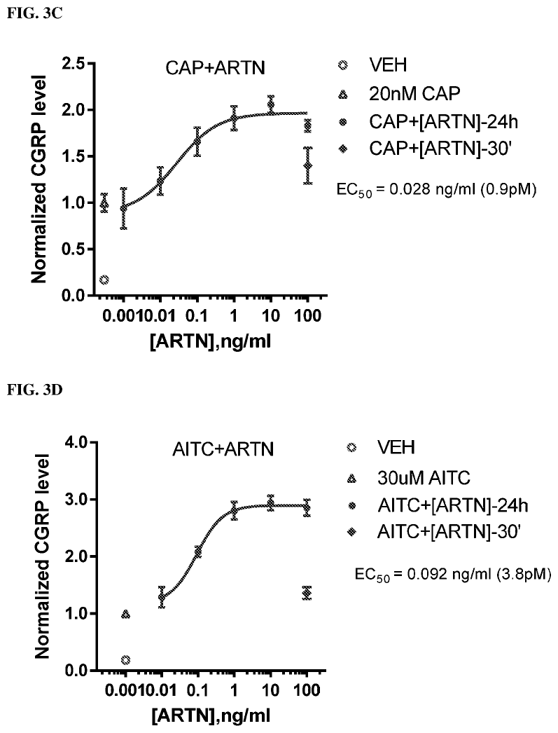

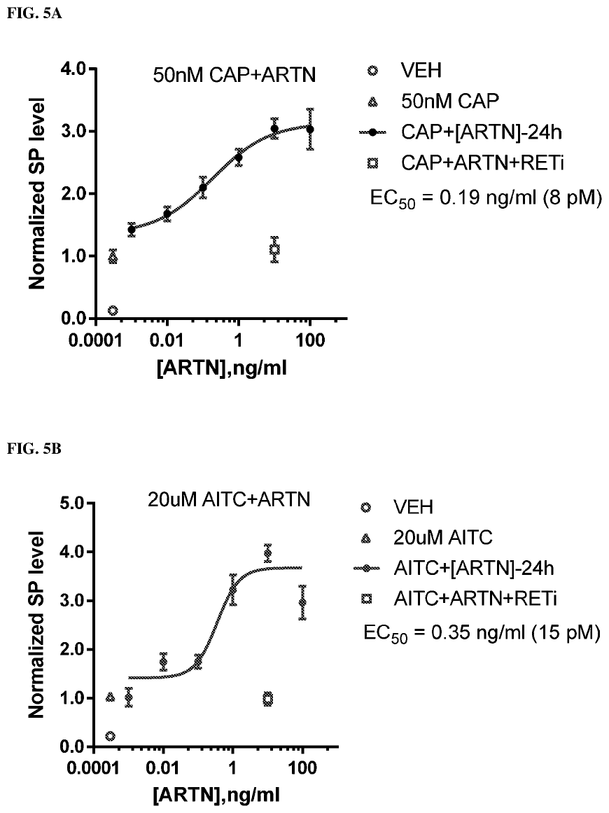

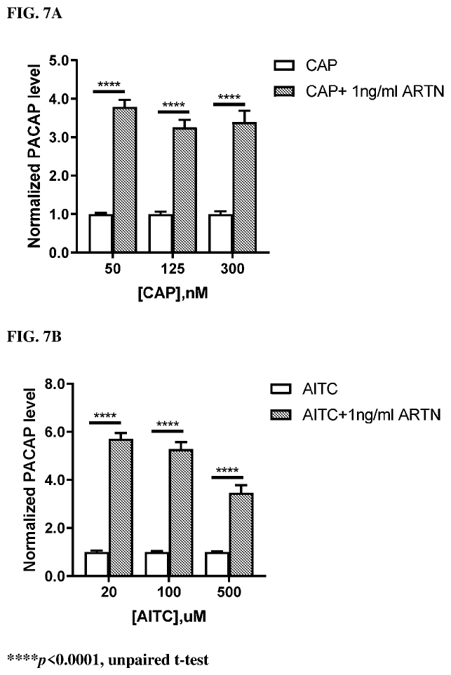

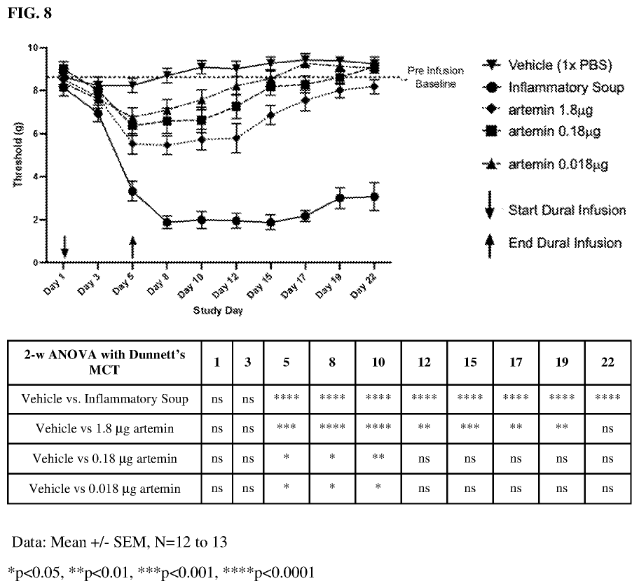

CGRP release in response to a concentration range of CAP, in the absence and presence of artemin (ARTN), was examined (FIGS. 3A to 3D). As shown in FIG. 3A and FIG. 3B, 10 ng/ml rat artemin impacts the dose-response relationship of CAP and AITC on CGRP release. Compared to dose-response curves of CAP or AITC alone, both treatment groups (30 min and 24 h artemin incubation) increased CAP or AITC-evoked CGRP release across a concentration range. The potency of CAP & AITC in the absence and presence of artemin were calculated as 50% effective concentrations (EC50). The EC50s (nM) for CAP, CAP+ARTN (30 min) and CAP+ARTN (24 h) are 21.63, 17.91 and 20.25, respectively. The EC50s (μM) for AITC, AITC+ARTN (30 min) and AITC+ARTN (24 h) are 17.83, 6.6 and 6.94, respectively. As shown in FIG. 3C and FIG. 3D, ARTN significantly potentiated CAP or AITC-evoked CGRP release with 24 h treatment in a dose-dependent manner. Following 24 hours of treatment, the EC50 for artemin-potentiation of CGRP release with CAP or AITC stimulation are 0.028 ng/ml (0.9 pM) and 0.092 ng/ml (3.8 pM), respectively. A similar protocol was used but for stimulating Substance P (in lieu of CGRP) release in collecting the data in FIGS. 5A and 5B. Samples for PACAP assay were prepared by treating cells with artemin for 48 hours, and three concentrations of TRPV1 agonist CAP and TRPA1 agonist AITC were used to evoke PACAP release in collecting the data in FIGS. 7A and 7B. These results demonstrates that in addition to CGRP, artemin increases other migraine-associated neuropeptides Substance P and PACAP mediated by TRPV1 and TRPA1 channel activation.

Compared to the dose-response curve of CAP or AITC alone group, treatment with artemin (10 ng/ml) for both 30 min and 24 h increased CAP and AITC-evoked CGRP release at multiple concentrations (FIGS. 3A and 3B). Then CAP and AITC concentrations were fixed (˜EC50) and artemin dose-response impact on stimulated CGRP release in the range of 0.001 and 100 ng/ml was examined. Artemin showed similar potency for CAP and AITC-stimulated CGRP release and reached maximal effect at 1 ng/ml (FIGS. 3C and 3D).

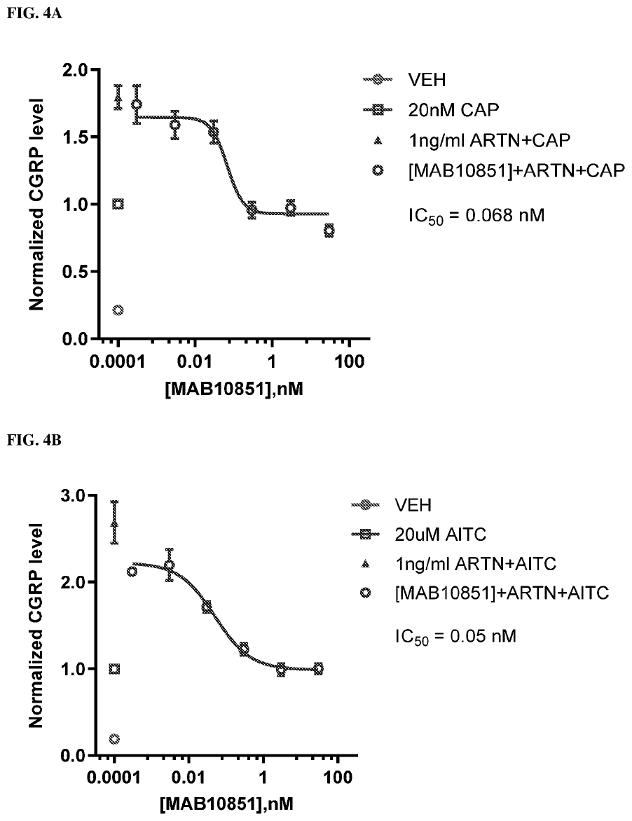

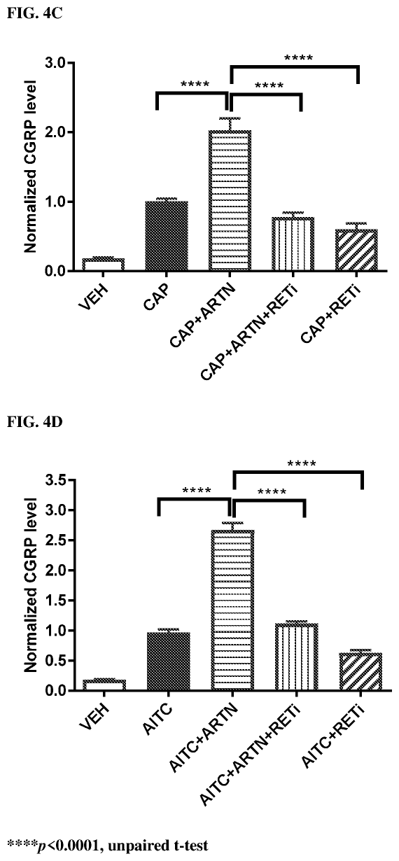

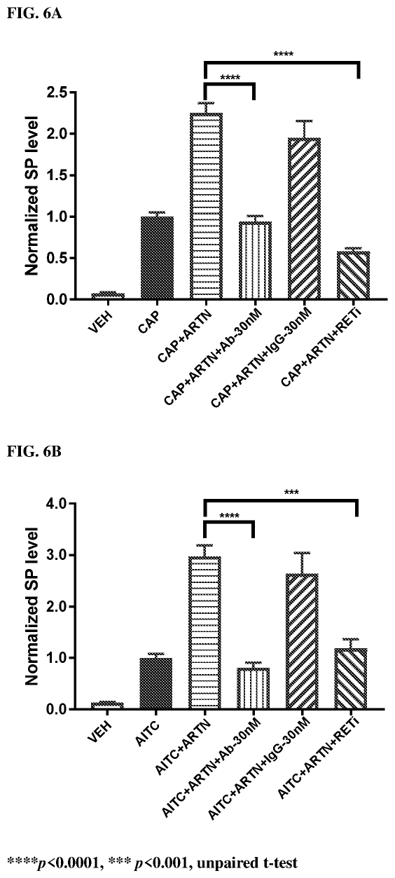

Artemin effect was verified by applying an anti-artemin antibody and RET inhibitor (FIGS. 4A to 4D). The anti-artemin antibody of mouse origin dose-dependently blocked artemin-increased CGRP release with stimulation of CAP and AITC. The IC50 for artemin antibody activity on blocking increased CGRP release with CAP and AITC stimulation are 0.068 nM and 0.05 nM, respectively (FIGS. 4A and 4B). As a positive control, the RET inhibitor (RETi, 100 nM) also completely inhibited ARTN-potentiated CAP/AITC-evoked CGRP release (FIGS. 4C and 4D), indicating that RET receptor is involved in the artemin-TRP channel-CGRP pathway and supporting the antagonism directionality for treating or preventing migraine and trigeminal neuralgia and other indications disclosed herein. Unpaired t-test statistical analysis was conducted for two-group comparison, and p<0.05 was considered as significant difference. A similar protocol was used but blocking Substance P (in lieu of CGRP) release in collecting the data in FIGS. 6A and 6B. IgG isotype control had no impact on SP release.

In addition, artemin impact on TG neuron sensitization was further validated in vivo by locally injecting rat artemin protein onto dura area in rat. Daily administration of three different amounts of artemin (0.018 μg, 0.18 μg and 1.8 μg) for five consecutive days significantly increased periorbital sensitivity in a dose-dependent manner compared to vehicle control group during the first two weeks (FIG. 8). This in vivo finding further supports the role of artemin pathway in contributing to trigeminal sensory pathway sensitization and headache pain.

Collectively, the current in vitro target validation data has demonstrated that artemin potentiates the release of multiple migraine-associated neuropeptides including CGRP, PACAP and SP from rodent trigeminal neurons by external triggers such as nociceptive channel agonists CAP and AITC causing higher risk of migraine attack. An anti-artemin antibody can bring the CGRP and SP level back to control level. This indicates that administering a neutralizing Artemin antibody to patients in need should provide a therapeutic benefit for migraine and trigeminal neuralgia and other indications disclosed herein and the treatment and prevention thereof.

SEQUENCE LISTING

Human Artemin:
SEQ ID NO: 1 (19R isoform) 220 aa
MELGLGGLSTLSHCPWPRRQPALWPTLAALALLSSVAEASLGSAPRSPA
PREGPPPVLASPAGHLPGGRTARWCSGRARRPPPQPSRPAPPPPAPPSA
LPRGGRAARAGGPGSRARAAGARGCRLRSQLVPVRALGLGHRSDELVRF
RFCSGSCRRARSPHDLSLASLLGAGALRPPPGSRPVSQPCCRPTRYEAV
SFMDVNSTWRTVDRLSATACGCLG
SEQ ID NO: 2 (19Q isoform) 220 aa
MELGLGGLSTLSHCPWPRQQPALWPTLAALALLSSVAEASLGSAPRSPA
PREGPPPVLASPAGHLPGGRTARWCSGRARRPPPQPSRPAPPPPAPPSA
LPRGGRAARAGGPGSRARAAGARGCRLRSQLVPVRALGLGHRSDELVRF
RFCSGSCRRARSPHDLSLASLLGAGALRPPPGSRPVSQPCCRPTRYEAV
SFMDVNSTWRTVDRLSATACGCLG
SEQ ID NO: 3 (predicted coding sequence)
ATGGAACTTGGACTTGGAGGCCTCTCCACGCTGTCCCACTGCCCCTGGC
CTAGGCAGCAGCCTGCCCTGTGGCCCACCCTGGCCGCTCTGGCTCTGCT
GAGCAGCGTCGCAGAGGCCTCCCTGGGCTCCGCGCCCCGCAGCCCTGCC
CCCCGCGAAGGCCCCCCGCCTGTCCTGGCGTCCCCCGCCGGCCACCTGC
CGGGGGGACGCACGGCCCGCTGGTGCAGTGGAAGAGCCCGGCGGCCGCC
GCCGCAGCCTTCTCGGCCCGCGCCCCCGCCGCCTGCACCCCCATCTGCT
CTTCCCCGCGGGGGCCGCGCGGCGCGGGCTGGGGGCCCGGGCAGCCGCG
CTCGGGCAGCGGGGGCGCGGGGCTGCCGCCTGCGCTCGCAGCTGGTGCC
GGTGCGCGCGCTCGGCCTGGGCCACCGCTCCGACGAGCTGGTGCGTTTC
CGCTTCTGCAGCGGCTCCTGCCGCCGCGCGCGCTCTCCACACGACCTCA
GCCTGGCCAGCCTACTGGGCGCCGGGGCCCTGCGACCGCCCCCGGGCTC
CCGGCCCGTCAGCCAGCCCTGCTGCCGACCCACGCGCTACGAAGCGGTC
TCCTTCATGGACGTCAACAGCACCTGGAGAACCGTGGACCGCCTCTCCG
CCACCGCCTGCGGCTGCCTGGGCTGA

1. A method of preventing or treating migraine a subject in need thereof, comprising administering to the subject an inhibitor of human artemin.

2. The method of claim 1, wherein the human artemin comprises the sequence set out in SEQ ID NO: 1.

3. The method of claim 1, wherein the human artemin comprises the sequence set out in SEQ ID NO: 2.

4. The method of claim 1, wherein the subject is a human subject.

5. The method of claim 4, wherein the subject is a human subject detected to having an Artemin comprising the sequence set out in SEQ ID NO: 1.

6. The method of claim 1, wherein the use comprises administering is a subcutaneous administration.

7. The method of claim 1, wherein the inhibitor is present in an aqueous solution.

8. The method of claim 1, wherein the inhibitor is a humanized monoclonal antibody.

9. The method of claim 1, wherein the inhibitor is a bispecific antibody targeting CGRP and the human artemin.

10. The method of claim 1, wherein the inhibitor is a bispecific antibody targeting Substance P and the human artemin.

11. The method of claim 1, wherein the use method is the prevention of migraine.

12. The method of claim 11, wherein a reduction in mean monthly migraine-occurrence days is at least 10% greater for a population of patients administered with the inhibitor compared to a placebo or a therapeutic agent directly targeting CGRP.

13. The method of claim 11, wherein the 50% responder rate is higher for a population of patients administered with the inhibitor compared to a placebo or a therapeutic agent directly targeting CGRP.

14. The method of claim 1, wherein the use method is the treatment of migraine.

15. The method of claim 14, wherein a percentage of patients that are pain free 2 hours after administration of the inhibitor is at least 10% higher for a population of patients administered with the inhibitor compared to a population of patients administered with a placebo or a therapeutic agent directly targeting CGRP.

16. The method of claim 14, wherein a percentage of patients that are pain free 2 hours after administration of the inhibitor and do not use rescue medication or relapse within 24 hours after administration of the inhibitor is at least 10% higher compared to a population of patients administered with a placebo or a therapeutic agent directly targeting CGRP.

17. The method of claim 12, wherein the therapeutic agent directly targeting CGRP is erenumab, fremanezumab, galcanezumab, eptinezumab, ubrogepant, rimpegepant, or atogepant.

18. The method of claim 13, wherein the therapeutic agent directly targeting CGRP is erenumab, fremanezumab, galcanezumab, eptinezumab, ubrogepant, rimpegepant, or atogepant.

19. The method of claim 15, wherein the therapeutic agent directly targeting CGRP is erenumab, fremanezumab, galcanezumab, eptinezumab, ubrogepant, rimpegepant, or atogepant.

20. The method of claim 16, wherein the therapeutic agent directly targeting CGRP is erenumab, fremanezumab, galcanezumab, eptinezumab, ubrogepant, rimpegepant, or atogepant.


SEQUENCE LISTING (3 sequences)

SEQ ID NO: 1 - protein, 220 aa, Homo sapiens
MELGLGGLSTLSHCPWPRRQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 2 - protein, 220 aa, Homo sapiens
MELGLGGLSTLSHCPWPRQQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 3 - DNA, 663 bp, Homo sapiens
  source 1..663: mol_type = genomic DNA
atggaacttggacttggaggcctctccacgctgtcccactgcccctggcctaggcagcagcctgccctgtggcccaccctggccgctctggctctgctgagcagcgtcgcagaggcctccctgggctccgcgccccgcagccctgccccccgcgaaggccccccgcctgtcctggcgtcccccgccggccacctgccggggggacgcacggcccgctggtgcagtggaagagcccggcggccgccgccgcagccttctcggcccgcgcccccgccgcctgcacccccatctgctcttccccgcgggggccgcgcggcgcgggctgggggcccgggcagccgcgctcgggcagcgggggcgcggggctgccgcctgcgctcgcagctggtgccggtgcgcgcgctcggcctgggccaccgctccgacgagctggtgcgtttccgcttctgcagcggctcctgccgccgcgcgcgctctccacacgacctcagcctggccagcctactgggcgccggggccctgcgaccgcccccgggctcccggcccgtcagccagccctgctgccgacccacgcgctacgaagcggtctccttcatggacgtcaacagcacctggagaaccgtggaccgcctctccgccaccgcctgcggctgcctgggctga